(19) United States
(12) Patent Application Publication
(10) Pub. No.: US 2024/0299082 A1
(43) Pub. Date: Sep. 12, 2024

(54) APPARATUS AND METHOD FOR ABLATION WITH VARIABLE ELECTRODE SPACING AND FLUID MEDIUM

(71) Applicants: Acclarent, Inc., Irvine, CA (US); Biosense Webster (Israel) Ltd., Yokneam (IL)

(72) Inventors: Cesar Fuentes-Ortega, Pasadena, CA (US); Shubhayu Basu, Anaheim, CA (US); Henry F. Salazar, Pico Rivera, CA (US)

(21) Appl. No.: 18/433,795

(22) Filed: Feb. 6, 2024

Related U.S. Application Data
(60) Provisional application No. 63/450,111, filed on Mar. 6, 2023.

(51) Int. Cl. A61B 18/14; A61B 18/00

(52) U.S. Cl. CPC A61B 18/1477; A61B 2018/00654; A61B 2018/00755; A61B 2018/00875; A61B 2018/1425; A61B 2018/1467

(57) ABSTRACT

A method includes inserting a variable distance electrode assembly within a patient. A first electrode of the variable distance electrode assembly is placed adjacent to a targeted tissue area. A second electrode of the variable distance electrode assembly is placed adjacent to the targeted tissue area such that the first electrode and the second electrode are spaced from each other to define a first distance. The first and second electrodes are activated to apply electrical energy to the targeted tissue area. The first electrode is translated relative to the second electrode to define a second distance between each other while the first electrode and the second electrode continue to apply electrical energy to the targeted tissue area.

### PRIORITY

This application claims priority to U.S. Provisional Application No. 63/450,111, entitled “Apparatus and Method for Ablation with Variable Electrode Spacing and Fluid Medium,” filed on Mar. 6, 2023, the disclosure of which is incorporated by reference herein, in its entirety.

### BACKGROUND

Various medical fields (Electrophysiology, ENT, etc.) utilize tissue ablation, or other kinds of application of electrical energy to tissue, for treatment of medical conditions. One or more electrodes may be utilized to deliver ablation energy. In some clinical scenarios, radiofrequency (RF) energy may be utilized to ablate tissue. This may include contacting a surface of tissue with one or more electrodes, then activating the one or more electrodes to apply the RF energy to the tissue. In cases where one electrode is used, a ground pad may be placed in contact with skin or other tissue of the patient, and the one electrode that contacts the targeted tissue surface may apply monopolar RF energy to the targeted tissue surface. In cases where two or more electrodes are used, the two or more electrodes may be placed in contact with the targeted tissue surface and may be activated to apply bipolar RF energy to the targeted tissue surface. In either case, the RF energy may ablate the tissue to provide a desired therapeutic effect. Another modality for ablating tissue with electrical energy includes the use of pulsed field DC energy, such as in an irreversible electroporation (IRE) procedure.

FIG. 1 depicts non-limiting, illustrative, ENT anatomical structures where tissue may be ablated or otherwise treated with electrical energy. FIG. 1 depicts a left sagittal view of a portion of a patient's head, showing the nasal cavity (10), the frontal sinus (12), the sphenoid sinus (14), and the sphenoid bone (16). The nasal cavity (10) is bounded laterally by the nasal wall (18), which includes an inferior turbinate (20), a middle turbinate (22), and a superior turbinate (24). The vidian nerve (32) resides within the vidian (or “pterygoid”) canal (30), which is defined in part by the sphenoid bone (16) and is located posterior to the sphenoid sinus (14), approximately in alignment with the middle turbinate (22). The vidian nerve (32) is formed at its posterior end by the junction of the greater petrosal nerve (34) and the deep petrosal nerve (36); and joins at its anterior end with the pterygopalatine ganglion (38), which is responsible for regulating blood flow to the nasal mucosa. The posterior nasal nerves (40) join with the pterygopalatine ganglion (38) and extend through the region surrounding the inferior turbinate (20).

While instruments and methods for performing tissue ablation and otherwise applying electrical energy to tissue are known, it is believed that no one prior to the inventors has made or used the invention described in the appended claims.

### BRIEF DESCRIPTION OF THE DRAWINGS

The drawings and detailed description that follow are intended to be merely illustrative and are not intended to limit the scope of the invention as contemplated by the inventors.

FIG. 1 depicts a left sagittal view of a portion of a patient's head, showing details of certain anatomical structures;

FIG. 2 depicts a perspective view of an example of an instrument that may be used to apply electrical energy in a nasal cavity;

FIG. 3A depicts a perspective view of a distal portion of the shaft assembly of the instrument of FIG. 2, with a variable distance electrode assembly in a retracted position;

FIG. 3B depicts a perspective view of the distal portion of the shaft assembly of FIG. 3A, with the variable distance electrode assembly of FIG. 3A in an extended position;

FIG. 4A depicts an elevational side view of the distal portion of the shaft assembly of FIG. 3A spaced away from a targeted tissue surface, with the variable distance electrode assembly of FIG. 3A in the retracted position;

FIG. 4B depicts an elevational side view of the distal portion of the shaft assembly of FIG. 3A spaced away from the targeted tissue surface of FIG. 4A, with the variable distance electrode assembly of FIG. 3A in a first extended position;

FIG. 4C depicts an elevational side view of the distal portion of the shaft assembly of FIG. 3A, with the variable distance electrode assembly of FIG. 3A in the first extended position and advanced into engagement with the targeted tissue surface of FIG. 4A;

FIG. 4D depicts an elevational side view of the distal portion of the shaft assembly of FIG. 3A, with the variable distance electrode assembly of FIG. 3A in the first extended position and activated while engaged with the targeted tissue;

FIG. 4E depicts an elevational side view of the distal portion of the shaft assembly of FIG. 3A, with the variable distance electrode assembly of FIG. 3A extended into a second extended position and activated while engaged with the targeted tissue;

FIG. 5A depicts an elevational side view of the distal portion of the shaft assembly of FIG. 3A spaced away from a targeted tissue surface, with the variable distance electrode assembly of FIG. 3A in the retracted position;

FIG. 5B depicts an elevational side view of the distal portion of the shaft assembly of FIG. 3A spaced away from the targeted tissue surface of FIG. 5A, with the variable distance electrode assembly of FIG. 3A in the retracted position and a fluid delivery member in an extended position to deliver fluid o the targeted tissue surface;

FIG. 5C depicts an elevational side view of the distal portion of the shaft assembly of FIG. 3A spaced away from the targeted tissue surface of FIG. 5A and delivered fluid, with the variable distance electrode assembly of FIG. 3A in a first extended position;

FIG. 5D depicts an elevational side view of the distal portion of the shaft assembly of FIG. 3A, with the variable distance electrode assembly of FIG. 3A in the first extended position and advanced into engagement with the targeted tissue surface of FIG. 5A and delivered fluid;

FIG. 5E depicts an elevational side view of the distal portion of the shaft assembly of FIG. 3A, with the variable distance electrode assembly of FIG. 3A in the first extended position and activated while engaged with the targeted tissue surface of FIG. 5A and delivered fluid;

FIG. 5F depicts an elevational side view of the distal portion of the shaft assembly of FIG. 3A, with the variable distance electrode assembly of FIG. 3A extended into a second extended position and activated while engaged with the targeted tissue surface of FIG. 5A and delivered fluid;

FIG. 6 depicts a perspective view of a distal portion of a shaft assembly having an alternative variable distance electrode assembly;

FIG. 7 depicts a perspective view of a distal portion of a shaft assembly having another alternative variable distance electrode assembly;

FIG. 8A depicts an elevational side view of the distal portion of the shaft assembly of FIG. 7 spaced away from a targeted tissue surface, with the variable distance electrode assembly of FIG. 7 in the retracted position;

FIG. 8B depicts an elevational side view of the distal portion of the shaft assembly of FIG. 7 spaced away from the targeted tissue surface of FIG. 8A, with the variable distance electrode assembly of FIG. 7 in a partially extended position such that a fluid delivery member is an extended position;

FIG. 8C depicts an elevational side view of the distal portion of the shaft assembly of FIG. 7 spaced away from the targeted tissue surface of FIG. 8A, with the variable distance electrode assembly of FIG. 7 in the partially extended position such that the fluid delivery member is an extended position to deliver fluid onto the targeted tissue surface;

FIG. 8D depicts an elevational side view of the distal portion of the shaft assembly of FIG. 7 spaced away from the targeted tissue surface of FIG. 8A, with the variable distance electrode assembly of FIG. 7 in the partially extended position such that the fluid delivery member is an extended position with a first electrode in contact with fluid on the targeted tissue surface;

FIG. 8E depicts an elevational side view of the distal portion of the shaft assembly of FIG. 7 spaced away from the targeted tissue surface of FIG. FIG. 8A, with the variable distance electrode assembly of FIG. 7 in an extended position such that both the fluid delivery member and a probe are is an extended position with a first and second electrodes in contact with fluid on the targeted tissue surface;

FIG. 8F depicts an elevational side view of the distal portion of the shaft assembly of FIG. 7 spaced away from the targeted tissue surface of FIG. 8A, with the variable distance electrode assembly of FIG. 7 in an extended position such that both the fluid delivery member and a probe are is an extended position with the first and second electrodes of FIG. 8E activated and in contact with fluid on the targeted tissue surface;

FIG. 9 depicts a perspective view of a distal portion of a shaft assembly having another alternative electrode assembly;

FIG. 10 depicts a perspective view of a distal portion of a shaft assembly having another alternative electrode assembly;

FIG. 11 depicts a perspective view of a distal portion of a shaft assembly having another alternative variable distance electrode assembly;

FIG. 12 depicts an elevational side view of two shaft assemblies, each containing a selected portion of an alternative variable distance electrode assembly;

FIG. 13 depicts a perspective view of an alternative shaft assembly and alternative variable distance electrode assembly, with the variable distance electrode assembly in an extended position; and

FIG. 14 depicts a front plan view of the shaft assembly and variable distance electrode assembly of FIG. 13.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

For clarity of disclosure, the terms “proximal” and “distal” are defined herein relative to a surgeon, or other operator, grasping a surgical instrument having a distal surgical end effector. The term “proximal” refers to the position of an element arranged closer to the surgeon, and the term “distal” refers to the position of an element arranged closer to the surgical end effector of the surgical instrument and further away from the surgeon. Moreover, to the extent that spatial terms such as “upper,” “lower,” “vertical,” “horizontal,” or the like are used herein with reference to the drawings, it will be appreciated that such terms are used for illustrative description purposes only and are not intended to be limiting or absolute. In that regard, it will be understood that surgical instruments such as those disclosed herein may be used in a variety of orientations and positions not limited to those shown and described herein.

As used herein, the terms “about” and “approximately” for any numerical values or ranges indicate a suitable dimensional tolerance that allows the part or collection of components to function for its intended purpose as described herein.

### I. Example of RF Ablation Instrument with Variable Electrode Spacing

As mentioned above, one or more electrodes may be utilized to deliver ablation energy (e.g. RF energy) onto targeted anatomical structures. In instances where two electrodes are utilized (e.g., to apply bipolar RF energy), ablation energy may travel through tissue between a first electrode and a second electrode in order to suitably ablate the tissue. The distance between electrodes may help define the area and/or depth of tissue to be ablated. In some instances, it may be desirable to customize the area/depth of a tissue ablation and/or have greater control of the tissue ablation process. For example, in some instances, an anatomical structure having a first tissue area to be ablated may be a first size; while a second tissue area to be ablated may be a second, different, size. Therefore, it may be desirable to control the spacing between two electrodes being used to cooperatively ablate tissue. Further, in order to provide for greater control of the tissue ablation process, it may be desirable to gradually increase the distance between the two electrodes while ablating tissue, thereby creating a continuously elongating ablation line and/or other pattern or form that is customizable depending on the desired ablation dimensions.

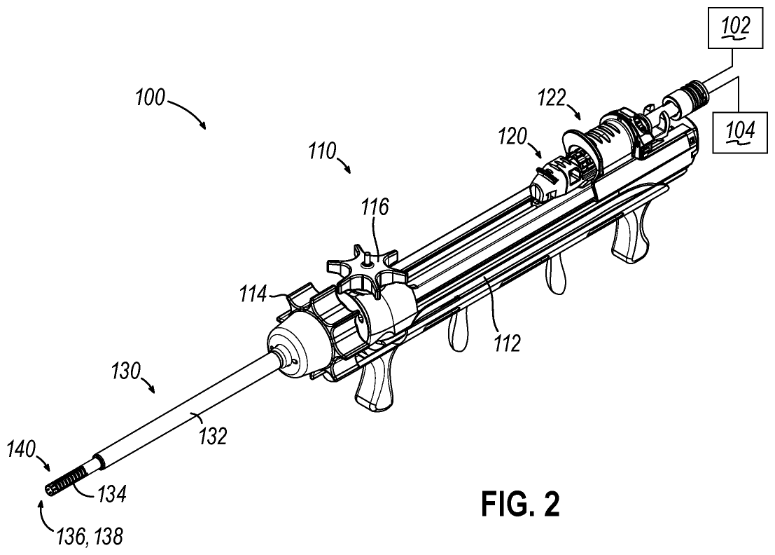

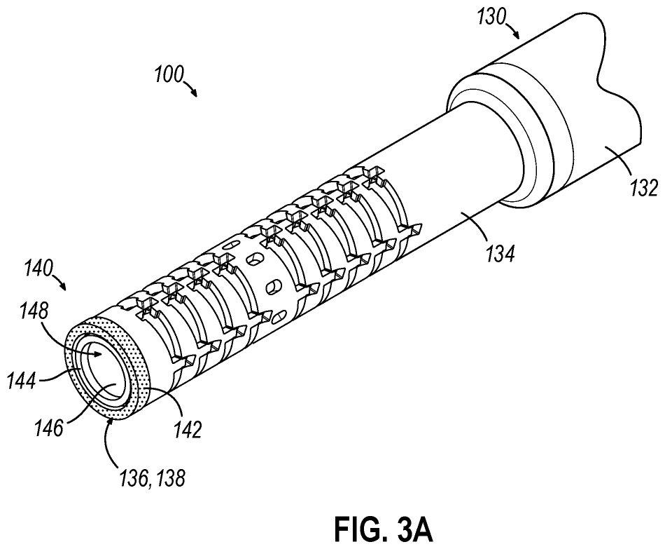

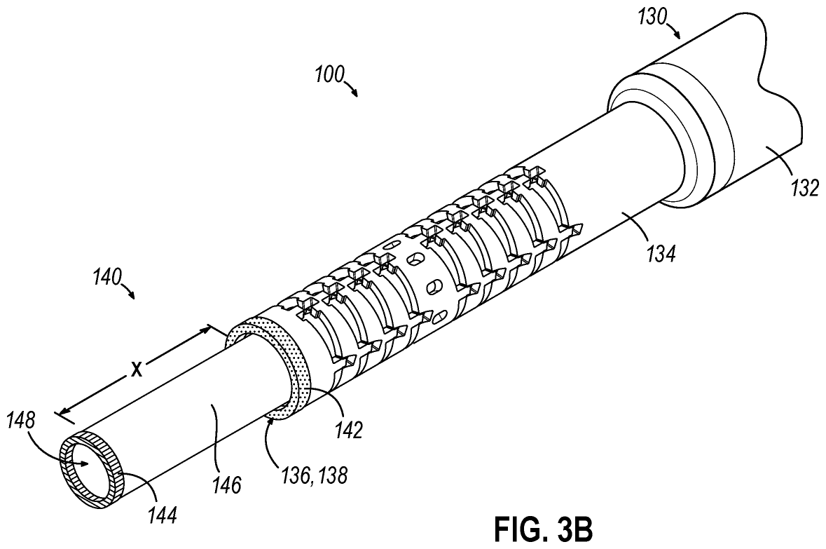

FIGS. 2-3B show an example of an instrument (100) that may be used to deliver RF energy to tissue. For instance, instrument (100) may be used to ablate a nerve (e.g., the posterior nasal neve (40)); ablate a turbinate (e.g., any of turbinates (20, 22, 24)); or ablate, electroporate (e.g., to promote absorption of therapeutic agents, etc.), or apply resistive heating to any other kind of anatomical structure in the head of a patient. As will be described in greater detail below, instrument (100) includes variable distance electrode assembly (140) that allows for customizable displacement between a stationary electrode (142) and a movable electrode (144), thereby allowing greater control of tissue ablation.

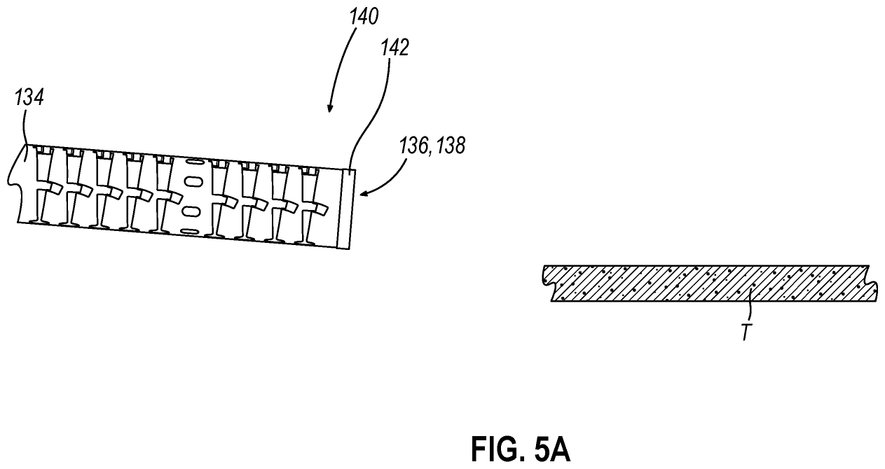

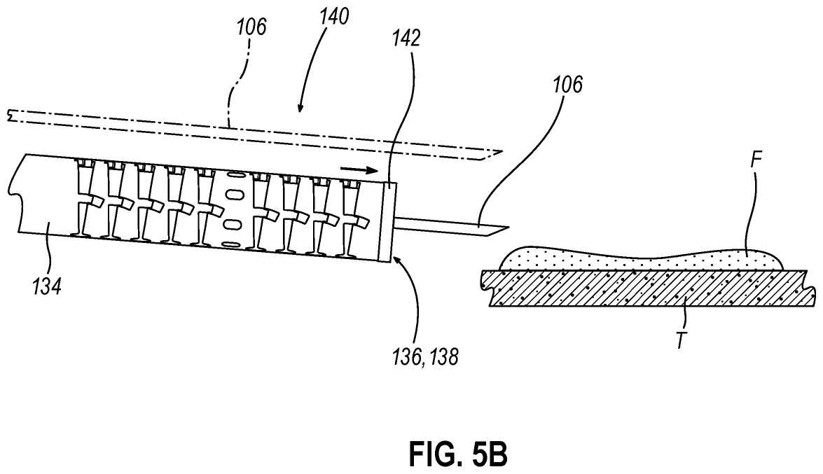

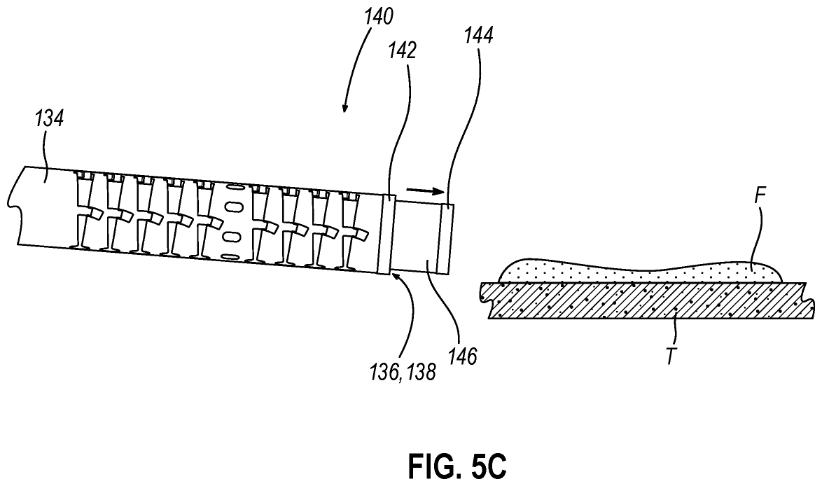

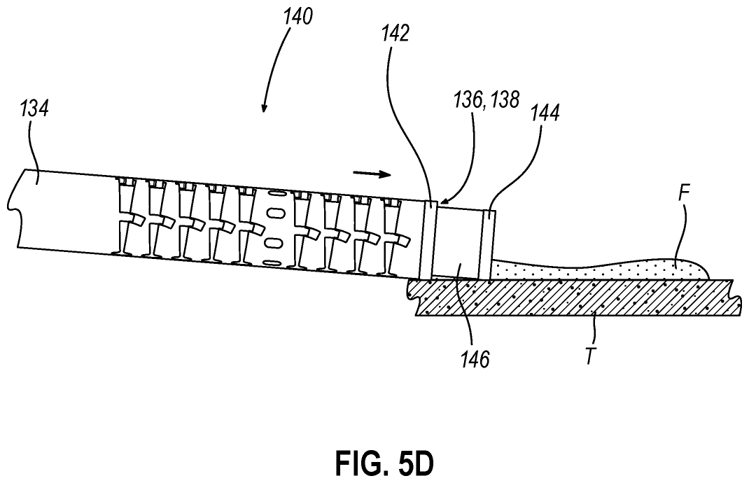

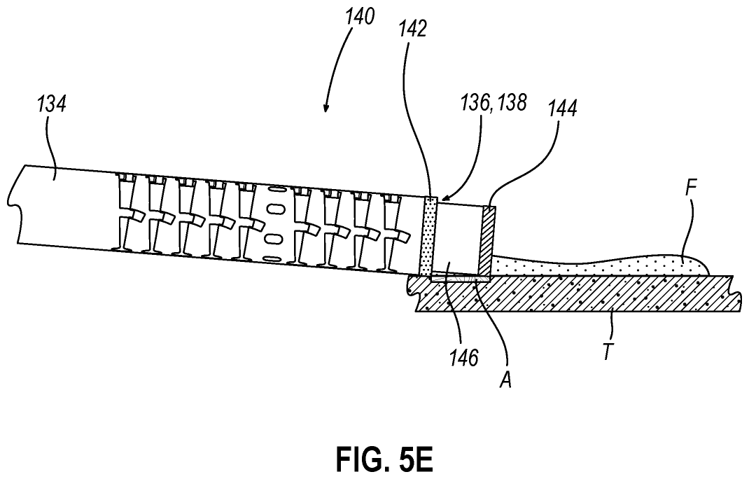

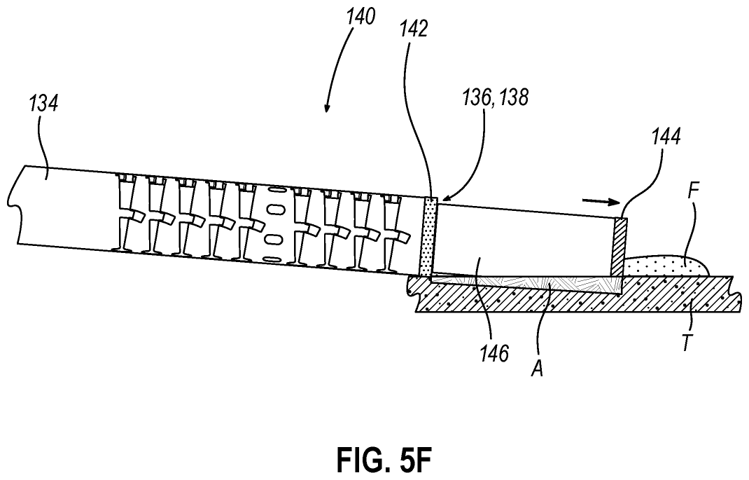

Instrument (100) of this example includes a handle assembly (110), a shaft assembly (130), and variable distance electrode assembly (140). Instrument (100) is coupled with an RF generator (102), which is operable to generate RF electrosurgical energy for delivery to tissue via electrodes (142, 144), as will be described in greater detail below. In some instances, instrument (100) may include a fluid delivery member (106) (see FIG. 5B) slidably disposed within shaft assembly (130) and in fluid communication with a fluid source (104) (see FIG. 2). Fluid delivery member (106) may include any suitable structure as would be apparent to one skilled in the art in view of the teachings herein. For example, fluid delivery member (106) may comprise a needle configured to suitably penetrate anatomical structures to reach a desired area. Fluid delivery member (106) may be formed of any suitable material and any suitable geometry as would be apparent to one skilled in the art in view of the teachings herein.

A. Overview of Illustrative Handle assembly and Shaft Assembly

Handle assembly (110) of this example includes a body (112), a first slider (120), and a second slider (122). Body (112) is sized and configured to be grasped and operated by a single hand of an operator, such as via a power grip, a pencil grip, or any other suitable kind of grip. Each slider (120, 122) is operable to translate longitudinally relative to body (112). Sliders (120, 122) are operable to translate independently relative to each other in some versions. First slider (120) is operatively coupled to an actuation body (146) of variable distance electrode assembly (140) such that actuation of first slider (120) relative to body (112) drives movement of actuation body (146) relative to flexible portion (134) of shaft assembly (130). In instances where a fluid delivery member (106) is utilized, second slider (122) may be operatively coupled to fluid delivery member (106) such that actuation of second slider (122) relative to body (112) drives movement of fluid delivery member (106) relative to shaft assembly (130).

Shaft assembly (130) of the present example includes a rigid portion (132), a flexible portion (134) distal to rigid portion (132), and an open distal end (136). A pull-wire (not shown) is coupled with flexible portion (134) and with a deflection control knob (116) of handle assembly (110). Deflection control knob (116) is rotatable relative to body (112), about an axis that is perpendicular to the longitudinal axis of shaft assembly (130), to selectively retract the pull-wire proximally. As the pull-wire is retracted proximally, flexible portion (134) bends and thereby deflects distal end (136) laterally away from the longitudinal axis of rigid portion (132). Deflection control knob (116), the pull-wire, and flexible portion (134) thus cooperate to impart steerability to shaft assembly (130). By way of example only, such steerability of shaft assembly (130) may be provided in accordance with at least some of the teachings of U.S. Pat. Pub. No. 2021/0361912, entitled “Shaft Deflection Control Assembly for ENT Guide Instrument,” published Nov. 25, 2021, the disclosure of which is incorporated by reference herein, in its entirety. Other versions may provide some other kind of user input feature to drive steering of flexible portion (134), instead of deflection control knob (116). In some alternative versions, deflection control knob (116) is omitted, and flexible portion (134) is malleable. In still other versions, the entire length of shaft assembly (130) is rigid.

Shaft assembly (130) is also rotatable relative to handle assembly (110), about the longitudinal axis of rigid portion (132). Such rotation may be driven via rotation control knob (114), which is rotatably coupled with body (112) of handle assembly (110). Alternatively, shaft assembly (130) may be rotated via some other form of user input; or may be non-rotatable relative to handle assembly (110). It should also be understood that the example of handle assembly (110) described herein is merely an illustrative example. Shaft assembly (130) may instead be coupled with any other suitable kind of handle assembly or other supporting body.

While not shown, instrument (100) may also include one or more position sensors that are operable to generate signals indicative of the position of distal end (136), or some other component of instrument (100), in three-dimensional space. Such a position sensor may be integrated directly into shaft assembly (130) or elsewhere into instrument. In addition, or in the alternative, such a position sensor may be integrated into a guidewire or other component that is disposed in shaft assembly (130). Such a position sensor may take the form of one or more coils that generate signals in response to the presence of an alternating magnetic field. The position data generated by such position signals may be processed by a system that provides a visual indication to the operator to show the operator where the distal end (136), or some other component of instrument (100), is located within the patient in real time. Such a visual indication may be provided as an overlay on one or more preoperatively obtained images (e.g., CT scans) of the patient's anatomy. Such position sensing and navigation capabilities may be provided in accordance with at least some of the teachings of the various references cited herein.

### B. Illustrative Variable Distance Electrode Assembly

As best shown between FIGS. 3A-3B, variable distance electrode assembly (140) includes a stationary electrode (142), a movable electrode (144), and an actuating body (146) slidably disposed within lumen (138) of shaft assembly (130). Stationary electrode (142) is attached to open distal tip (136) of shaft assembly (130). Stationary electrode (142) is in electrical communication with RF generator (102) such that RF generator (102) may suitably activate stationary electrode (142) in accordance with the description herein. Stationary electrode (142) may be in communication with RF generator (102) via a wire(s) and/or electrical traces extending proximally from electrode (142) and suitably coupled with RF generator (102).

Moveable electrode (144) is attached to the distal end of actuating body (146). As will be described in greater detail below, actuating body (146) may actuate relative to sheath assembly (130) in order to drive translation of moveable electrode (144) relative to stationary electrode (142), thereby controlling the distance (X) between electrodes (142, 144). Similar to stationary electrode (142), movable electrode (144) is in electrical communication with RF generator (102) such that RF generator (102) may suitably activate movable electrode (144) in accordance with the description herein. Movable electrode (144) may be in communication with RF generator (102) via a wire(s) and/or electrical traces extending proximally from electrode (144) and suitably coupled with RF generator (102).

RF generator (102) is configured to cooperatively activate electrodes (142, 144) in order to apply bipolar RF energy onto suitable tissue. Such bipolar RF energy may be utilized in order to ablate tissue located between electrodes (142, 144). In some versions, handle assembly (110) may include one or more activation buttons configured to instruct generator (102) to activate electrodes (142, 144) in accordance with the description herein. In some such versions, such button(s) is located on sliders (120, 122) and/or body (112).

In some instances, movable electrode (144) may be attached to other suitable sections of actuation body (146) as would be apparent to one skilled in the art in view of the teachings herein. For example, movable electrode (144) may be attached to an exterior surface of actuating body (146) that is somewhat proximal relative to the distal end of actuating body (146).

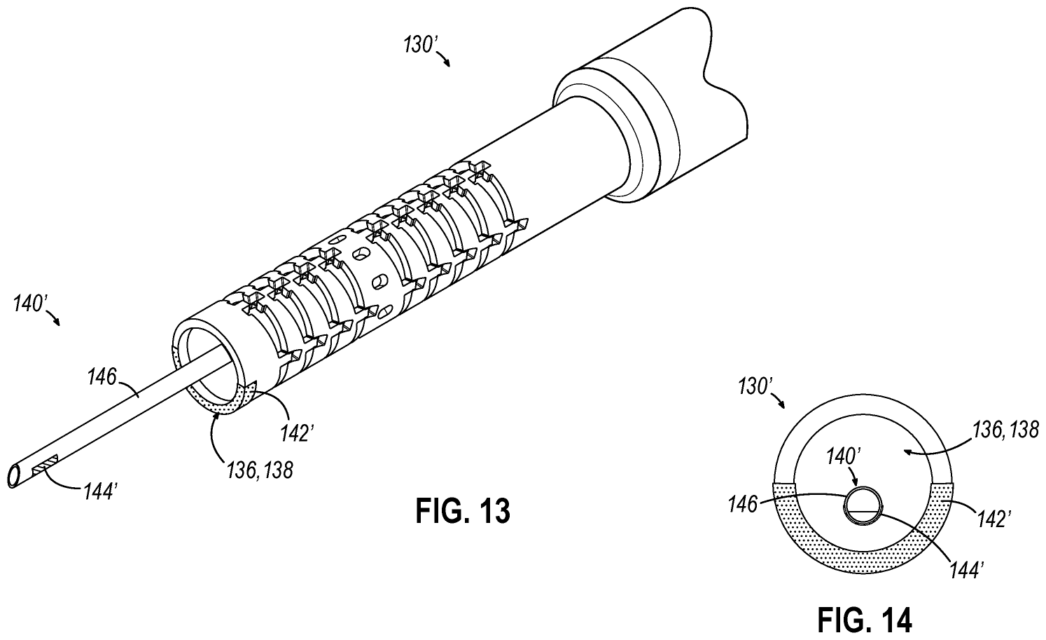

In the present example, stationary electrode (142) and moveable electrode (144) each include an annular shape. In some variations, electrodes (142, 144) may include any other suitable shape as would be apparent to one skilled in the art in view of the teachings herein. For example, one or both electrodes (142, 144) may include a semi-annular shape. Such a semi-annular shape may allow electrodes (142, 144) to better target tissue regions intended to be ablated in accordance with the description herein, while also inhibiting ablation of tissue/anatomical structures not intended to be ablated. Therefore, semi-annual electrodes (142, 144) may allow for control of tissue ablation in smaller anatomical regions where targeted tissue is close to non-targeted tissue. FIGS. 13-14 shows an alternative shaft assembly (130′) and variable distance electrode assembly (140′) that is substantially similar to shaft assembly (130) and variable distance electrode assembly (140) described herein, respectively, with differences described herein. Electrode assembly (140′) includes alternative electrodes (142′, 144′) rather than electrodes (142, 144). Stationary electrode (142′) is attached to open distal end (136) while movable electrode (144′) is attached to actuating body (146). Alternative electrodes (142′, 144′) are substantially similar to electrodes (142, 144) described above, except electrodes (142′, 144′) each form a semi-annular shape, which are half-rings in the current example. The semi-annular shape of electrode (142′, 144′) allows for electrodes (142′, 144′) to target tissue regions intended to be ablated in accordance with the description herein, while also inhibiting ablation of tissue/anatomical structures not intended to be ablated.

Actuating body (146) extends proximally within shaft assembly (130) and is operatively coupled to first slider (120). Therefore, as mentioned above, first slider (120) is operable to drive movement of actuating body (146) relative to shaft assembly (130). As best shown in FIG. 3B, actuating body (146) includes a suitable length such that first slider (120) may actuate a distal portion of actuating body (146) distally past open distal tip (136) of shaft assembly (130) up to a maximum distance (X). Maximum distance (X) may be any suitable distance as would be apparent to one skilled in the art in view of the teachings herein. It should be understood that actuating body (146) may also be actuated past open distal tip (136) any other suitable distance less than the maximum distance (X)

In instances where flexible portion (134) of shaft assembly (130) is configured to deflect relative to rigid portion (132) of shaft assembly, actuating body (146) may also include a sufficiently resilient and/or flexible sections that may also flex/bend to traverse flexible portion (134) in the deflected configuration as actuating body (146) is driven relative to shaft assembly (130) in accordance with the description herein. Actuating body (146) may be formed of any suitable material, or combination of materials, as would be apparent to one skilled in the art in view of the teachings herein.

In the current example, actuating body (146) also defines a working lumen (148). Working lumen (148) may be dimensioned to accommodate another surgical device, such as an endoscope, fluid delivery member (106), etc. In some instances, actuating body (146) does not define a working lumen (148) such that actuating body (146) is substantially solid.

Actuating body (146) may include any suitable dimensions as would be apparent to one skilled in the art in view of the teachings herein. For example, actuating body (146) may be dimensioned to define a suitable space between the interior surface defining working lumen (138) and the exterior surface of actuating body (146), where such a suitable space may accommodate other surgical devices. In such instances, the suitable space located between the exterior surface of actuating body (146) and the interior surface of working lumen (138) may slidably accommodate fluid delivery member (106) (see FIG. 5B). In some examples, a suitable space between the interior surface defining working lumen (138) and the exterior surface of actuating body (146) provide a fluid communication pathway. In such instances, such a fluid commutation pathway may be configured to deliver fluid onto a targeted tissue surface in accordance with the description herein.

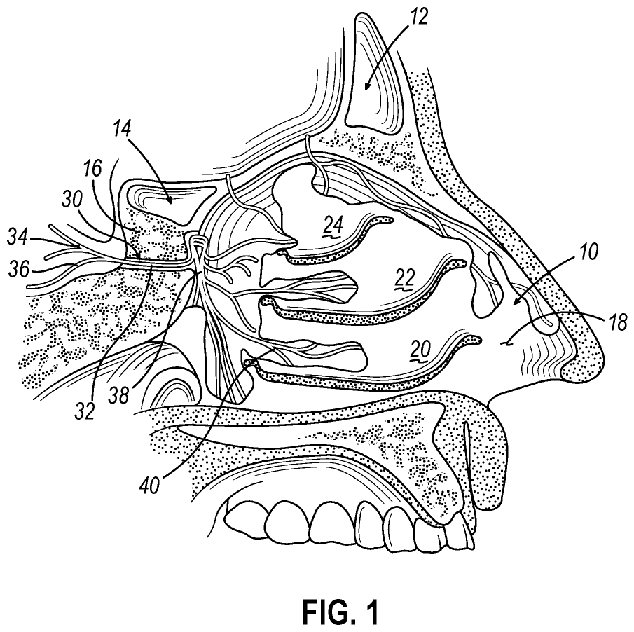

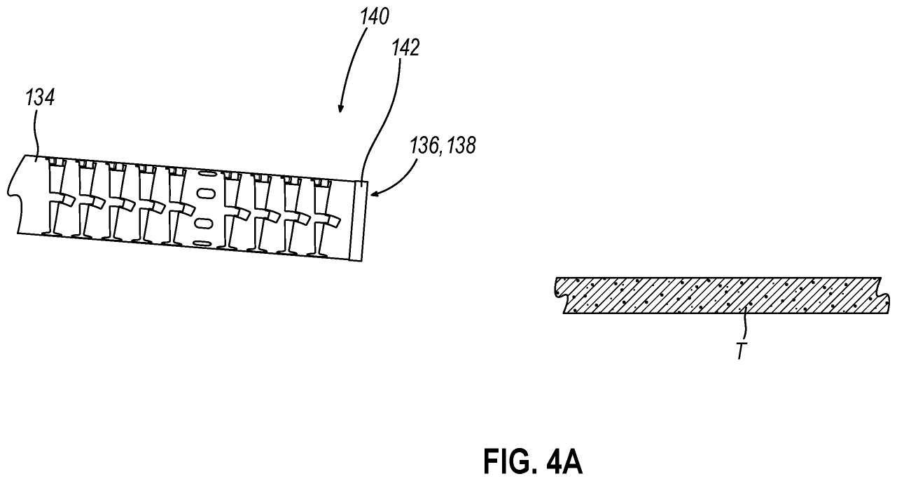

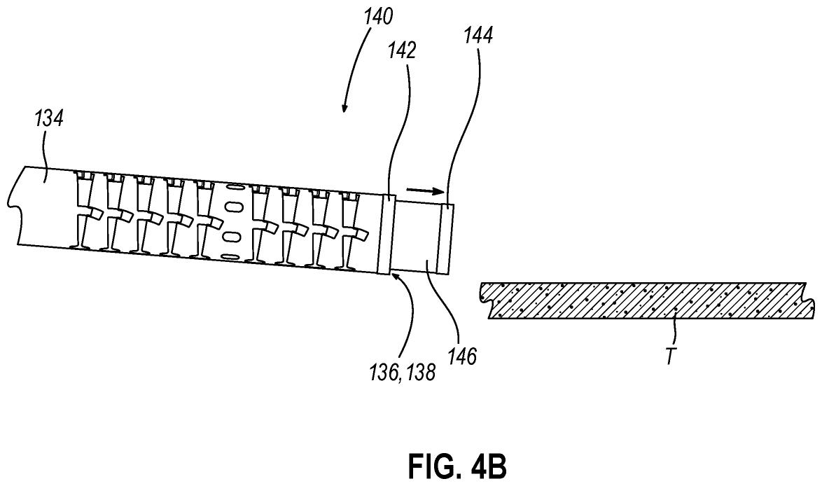

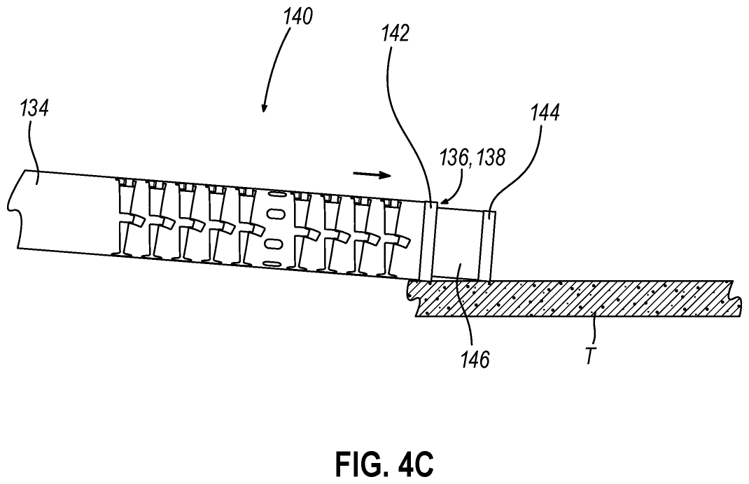

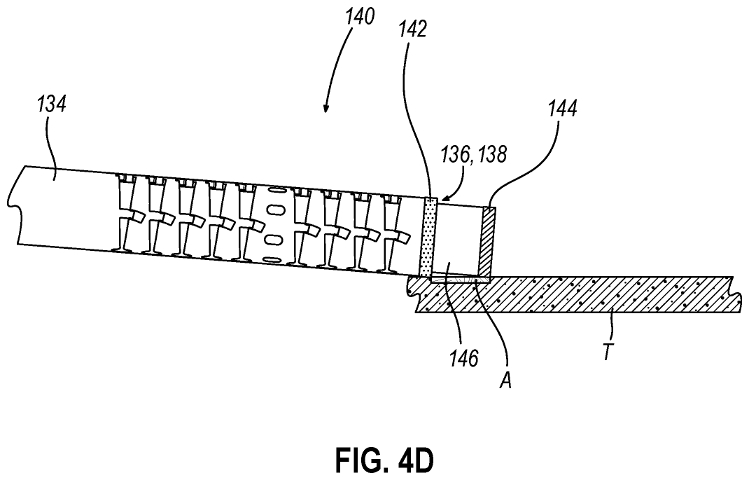

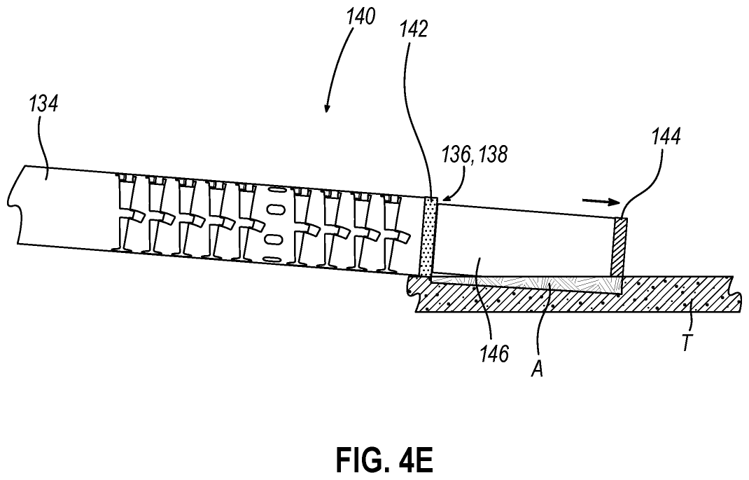

FIGS. 4A-4F show an example of use of variable distance electrode assembly (140) in order to controllably ablate a desired region of targeted tissue (T). First, an operator may suitably grasp and control instrument (100). Next, as shown in FIG. 4A, the operator may insert a portion of shaft assembly (130), including open distal tip (136), adjacent to targeted tissue (T) . In one non-limiting variation, shaft assembly (130) may be inserted through the nostril and within the nasal cavity (10) (see FIG. 1) of a patient.

Next, as shown in FIG. 4B, the operator may translate actuating body (146) out of open distal tip (136) such that electrodes (142, 144) are initially spaced away from each other. Next, as shown in FIG. 4C, the operator may advance shaft assembly (130) and actuating body (146) together until electrodes (142, 144) are in contact with targeted tissue (T). It should be understood that at the moment shown in FIG. 4D, the operator may further adjust the spacing between electrodes (142, 144) in accordance with the description herein. Therefore, by adjusting the spacing, the operator may better control the amount of targeted tissue (T) that is to be ablated. With targeted tissue (T) contacting electrode (142, 144), as shown in FIG. 4D, the operator may activate electrodes (142, 144) to deliver bipolar RF energy to tissue (T) located between electrodes (142, 144), thereby creating a controlled ablation region (A) of targeted tissue (T).

As mentioned above, in some instances, it may be desirable to further alter the dimensions of ablation region (A) of targeted tissue (T). If the operator desires to extend the area which targeted tissue (T) to be ablated, the operator may start to translate electrodes (142, 144) relative to each other while electrodes (142, 144) are still engaged with tissue, as shown in FIG. 4E. Therefore, providing variable moving electrodes (142, 144) may allow an operator to better control the ablated region (A) of tissue (T) during a surgical procedure.

In variations, as shown in FIG. 4E, the operator may translate actuating body (146) while shaft assembly (130) remains substantially stationary, thereby dragging electrode (144) distally across tissue (T). In other variations, the operator may actuate shaft assembly (130) while actuating body (146) remains substantially stationary, thereby dragging electrode proximally (142) across tissue (T). Yet in other variations, the operator may acuate both shafted assembly (130) and actuating body (146) relative to tissue (T), thereby dragging both electrodes (142, 144) simultaneously across tissue (T) in opposing directions.

In some variations, while moving electrodes (142, 144) as shown between FIGS. 4D-4E, the operator may drag at least one electrode (142, 144) across targeted tissue (T) while electrodes (142, 144) are still activated to deliver bipolar RF energy in accordance with the description herein. In other words, the operator may change the spatial distance between electrodes (142, 144) while electrodes are still activated and still engaged with tissue (T). Therefore, while moving electrode(s) (142, 144) across tissue (T), the operator may also be continuously ablating new portions of tissue (T), further controlling the portions of targeted tissue (T) being ablated. In some instances, the operator may stop activation of electrodes (142, 144), either momentarily or during the entire process of moving electrode(s) (142, 144) across tissue (T).

In some instances, the spacing between electrodes (142, 144) may become too great or the ablated tissue (T) may form too great electrical resistance in order to suitably maintain electrical continuity between electrodes (142, 144), while activated in accordance with the description herein, to suitably ablate tissue between electrodes (142, 144). Therefore, in some instances it may be desirable to further assist promoting or maintaining electrical continuity between electrodes (142, 144) in order to suitably ablate tissue (T) between electrodes (142, 144). FIGS. 5A-5F show an illustrative use of instrument (100) while communicating a conductive fluid (F) to the region of tissue were electrodes (142, 144) are located, to thereby promote or otherwise maintain electrical continuity between electrodes (142, 144), to thereby assist in tissue ablation.

As mentioned above, in some instances, second slider (122) is coupled to a fluid delivery member (106) in fluid communication with a fluid source (104) (see FIG. 2). Fluid delivery member (106) is sufficiently elongated in order to reach cavity (C) which target tissue (T) is located. As shown in FIG. 5A, the operator may access passageway (P) in a suitable manner as described above. Once access is achieved, as shown in FIG. 5B, the operator may flood cavity (C), or otherwise suitably distribute an electrically conductive fluid (F) (such as saline). In some instances, fluid delivery member (106) is housed within instrument (100). However, as shown in phantom, fluid delivery member (106) may be detached from the rest of instrument (100) and may be configured to access cavity (C) on its own, or with the assistance of another access instrument. In some variations where actuating body (146) defines a working lumen, fluid may be distributed via working lumen of actuating body (146). In such instances, actuating body (146) may be in fluid communication fluid source (104).

After electrically conductive fluid (F) is suitably delivered onto targeted tissue (T), the operator may remove fluid delivery member (106) from the site (as shown in FIG. 5C). Optionally, the operator may leave fluid delivery member (106) at the site of ablation and provide additional electrically conductive fluid (F) to the targeted site as needed. With a suitable amount of electrically conductive fluid (F) provided, as shown in FIGS. 5C-5F, the operator may utilize instrument (100) and variable distance electrode assembly (140) in a substantially similar manner as described above. Additionally, it should be understood that electrically conductive fluid (F) may be present between electrodes (142, 144) and in contact with tissue (T) in order to suitably assist in forming ablation region (A). Therefore, in instances where distance or already ablated tissue (A) is resisting further tissue ablation, fluid (F) may help assist further ablation. In some instances, electrodes (142, 144) may be utilized to sense impedance of targeted tissue (T). Delivery of electrically conductive fluid (F) may be automated based on the tissue impedance measurements provided by electrodes (142 144). For instance, if the sensed tissue impedance exceeds a threshold, this may automatically trigger delivery of electrically conductive fluid (F).

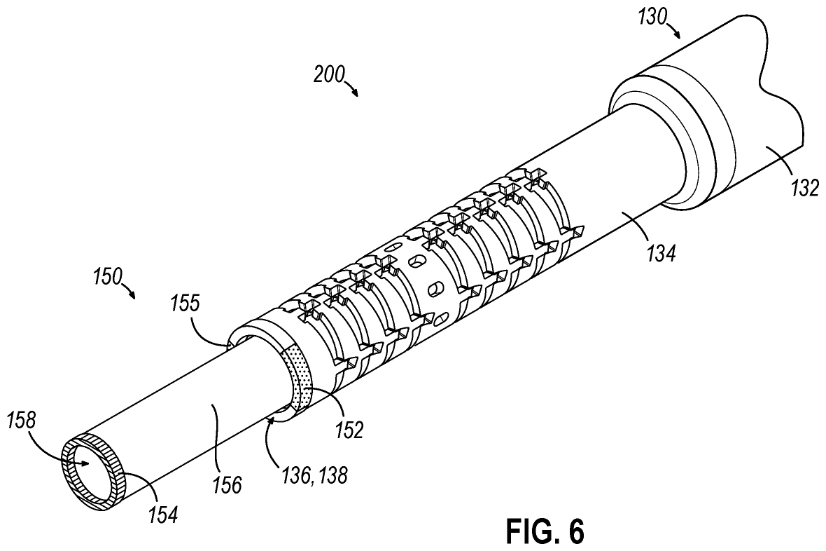

FIG. 6 shows an alternative instrument (200) that may be utilized in replacement of instrument (100) described above. Instrument (200) is substantially similar to instrument (100) described above, except with an alternative variable distance electrode assembly (150). Variable distance electrode assembly (150) includes a first stationary electrode (152), a movable electrode (155), and an actuation body (156) defining a working channel (158); which may be substantially similar to stationary electrode (142), movable electrode (142), actuating body (146), and working lumen (148) described above, with differences elaborated below.

First stationary electrode (152) is not annular in the example shown in FIG. 6, but only extends around a sector of the perimeter of open distal tip (136). A second stationary electrode (155) is also located on the perimeter of open distal tip (136), but is spaced away from first stationary electrode (152). Second stationary electrode (155) is in communication with RF generator (102) in similar fashion to first stationary electrode (152).

In some instances, when a more concentrated ablation area is desired, movable electrode (154) may be housed within the confines of shaft assembly (130). Further, movable electrode (154) may be deactivated in a “spot” ablation mode such that stationary electrodes (152, 155) are configured to apply bipolar RF energy to tissue. Therefore, stationary electrodes (152, 155) may have opposing poles when a concentrated “spot” ablation is desired, such that stationary electrodes (152, 155) cooperatively ablate tissue; while movable electrode (154) remains inactive.

When a variable distance ablation is desired, RF generator (102) may activate both stationary electrodes (152, 155) at a single polarity, thereby effectively treating both stationary electrodes (152, 155) as a single electrode which may be used in conjunction with movable electrode (154) to provide for a variable distance ablation. Therefore, in such configurations, the operator may utilize variable distance electrode assembly (150) in substantially the same manner as variable distance electrode assembly (140) described above.

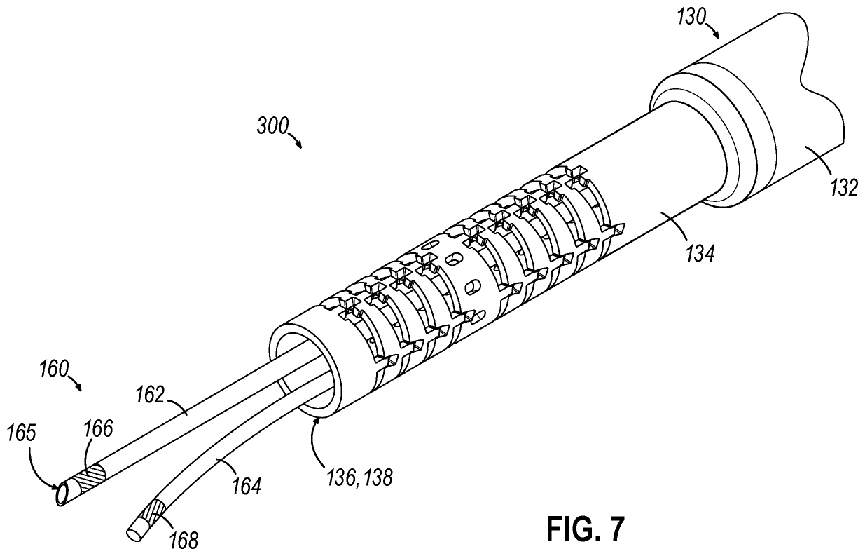

FIG. 7 shows an alternative instrument (300) that may be utilized in replacement to instrument (100, 200) described above. Therefore, instrument (300) is substantially similar to instrument (100, 200) described above, except with an alternative variable distance electrode assembly (160). In the present example, variable distance electrode assembly (160) includes a fluid delivery member (162) defining a fluid lumen (165), a probe body (164), a first electrode (166) associated with an exterior surface of fluid delivery member (162), and a second electrode (168) associated with an exterior surface of probe body (164).

Fluid delivery member (162) may be substantially similar to fluid delivery member (106) described above, with differences elaborated herein. Therefore, fluid delivery member (162) is in fluid communication with fluid source (104) such that fluid may travel from fluid source (104), through fluid delivery member (162) via fluid lumen (165), and exit open distal tip of fluid delivery member (162) in order to deliver a suitable fluid into a cavity (C) in accordance with the teachings herein. Fluid delivery member (162) extends through, and is slidably disposed within, shaft assembly (130) and includes an appropriate length in order to actuate distally past open distal tip (136) to suitably access targeted anatomical features. Second slider (122) may be operatively coupled to fluid delivery member (162) such that actuation of second slider (122) relative to body (112) drives movement of fluid delivery member (162) relative to shaft assembly (130).

In some instances, fluid delivery member (162) may include a needle. Fluid delivery member (162) may be formed of any suitable material and any suitable geometry as would be apparent to one skilled in the art in view of the teachings herein.

Fluid delivery member (162) also includes first electrode (166) disposed on a distal portion of fluid delivery member (162). First electrode (166) is in electrical communication with RF generator (102) such that RF generator (102) may suitably activate first electrode (166) in accordance with the description herein. First electrode (166) may be in communication with RF generator (102) via a wire(s) and/or electrical traces extending proximally from electrode (166) on fluid delivery member (162) and suitably attached to RF generator (102). Handle assembly (110), RF generator (102), or any other suitable structure may be utilized in order to activate first electrode (166) to ablate tissue.

Since first electrode (166) is disposed on fluid delivery member (162), first electrode (166) is also configured to actuate relative to sheath assembly (130) such that first electrode (166) may extend distally past open distal end (136) a suitable distance as would be apparent to one skilled in the art in view of the teachings herein. Therefore, first electrode (166) is capable of being positioned relative to shaft assembly (130) in order to selectively ablate tissue (T) in accordance with the description herein.

Similar to fluid delivery member (162), probe (164) extends through, and is slidably disposed within, shaft assembly (130) and includes an appropriate length in order to actuate distally past open distal tip (136) to suitably access targeted anatomical features. First slider (120) may be operatively coupled to probe (164) such that actuation of first slider (120) relative to body (112) drives movement of probe (164) relative to shaft assembly (130). Probe (164) may be formed of any suitable material and have any suitable geometry as would be apparent to one skilled in the art in view of the teachings herein. For example, probe (164) may be formed of a rigid material, a semi-rigid material, a pliable material, a resilient material, or any suitable combination of materials in order to suitably access a targeted tissue and suitably engage such targeted tissue in accordance with the teachings herein.

Probe (164) also includes second electrode (168) disposed on a distal portion of probe (164). Similar to first electrode (166), second electrode (168) is in electrical communication with RF generator (102) such that RF generator (102) may suitably activate second electrode (168) in accordance with the description herein. In particular, RF generator (102) is configured to activate both first electrode (166) and second electrode (168) in order to apply bipolar, RF energy, to tissue in order to ablate tissue. Second electrode (168) may be in communication with RF generator (102) via a wire(s) and/or electrical traces extending proximally from electrode (168) on probe (164) and suitably attached to RF generator (102). Handle assembly (110), RF generator (102), or any other suitable structure may be utilized in order to activate second electrode (168) to ablate tissue.

Since second electrode (168) is disposed on probe (164), second electrode (168) is also configured to actuate relative to sheath assembly (130) such that second electrode (168) may extend distally past open distal end (136) a suitable distance as would be apparent to one skilled in the art in view of the teachings herein. Therefore, second electrode (168) is capable of being positioned relative to shaft assembly (130) in order to selectively ablate tissue (T) in accordance with the description herein. With first and second electrodes (166, 168) configured to actuate relative to each other and sheath assembly (130), electrodes (166, 168) may be positioned various distances relative to each other in order to suitably ablate tissue, both prior to tissue ablation and during tissue ablation.

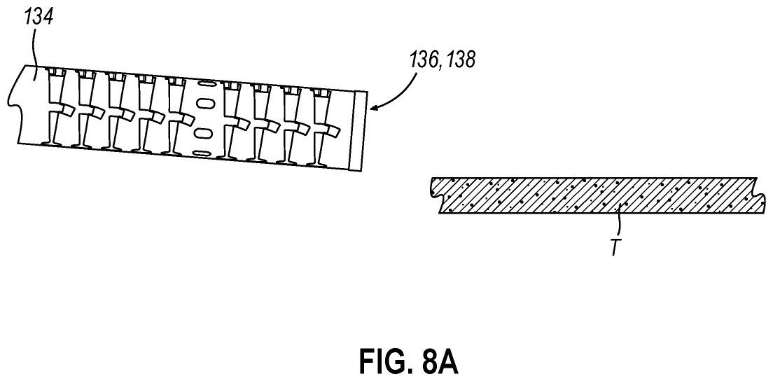

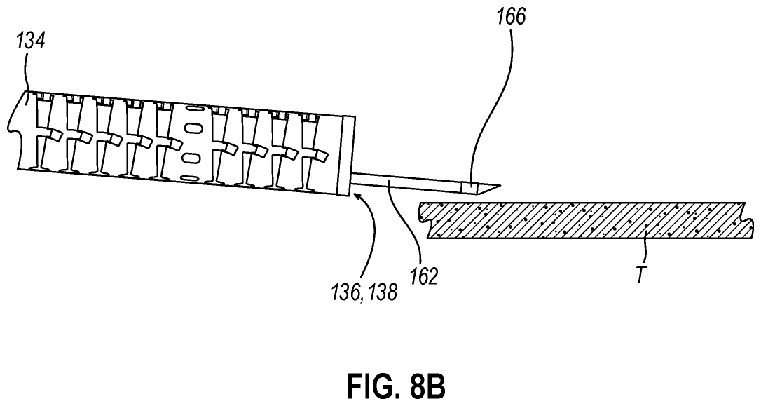

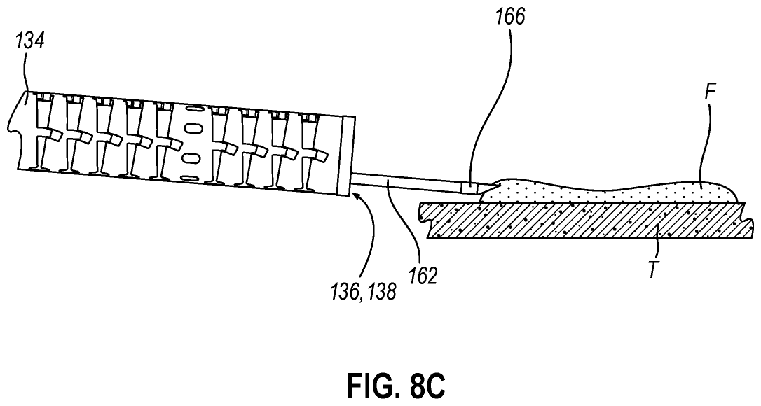

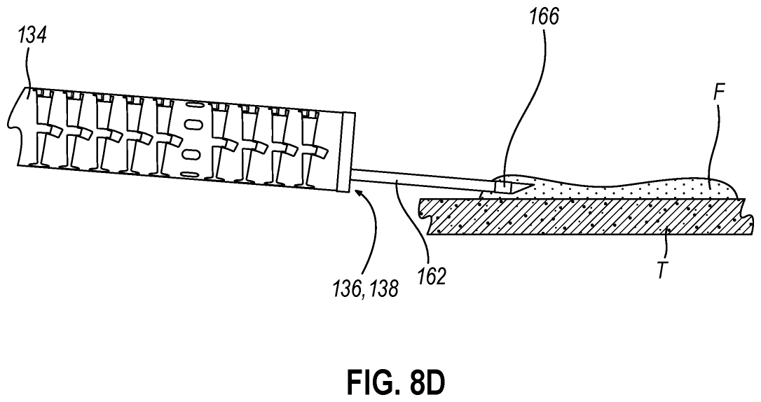

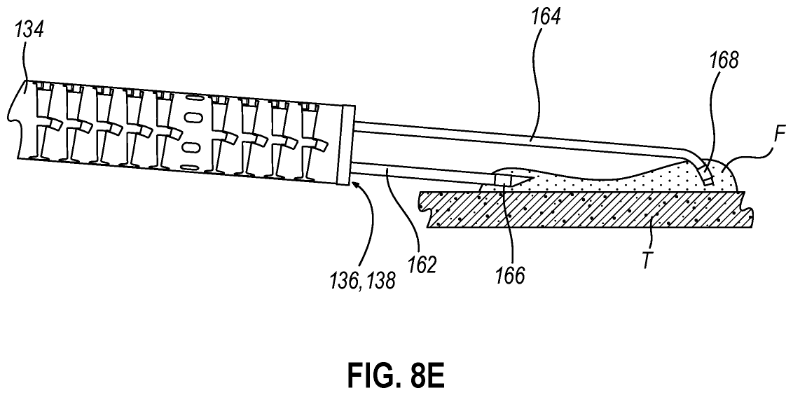

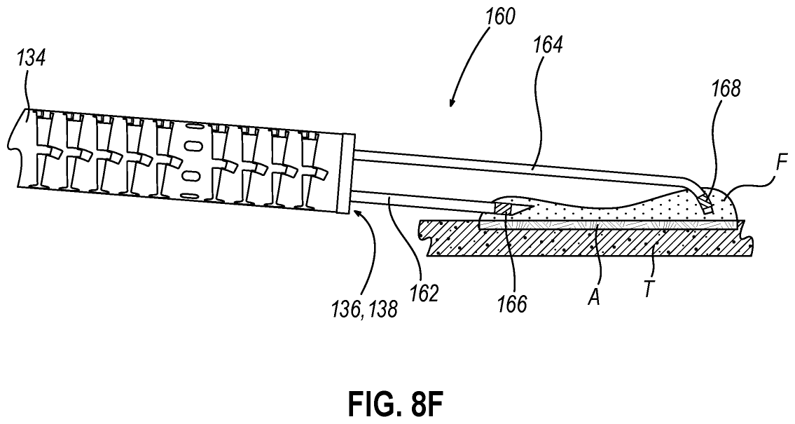

FIGS. 8A-8F show an example of use of variable distance electrode assembly (160) in order to controllably ablate a desired region of targeted tissue (T). First, an operator may suitably grasp and control instrument (300). Next, as shown in FIG. 8A, the operator may insert a portion of shaft assembly (130), including open distal tip (136), adjacent to targeted tissue (T). In one non-limiting variation, shaft assembly (130) may be inserted through the nostril and within the nasal cavity (10) (see FIG. 1) of a patient.

Next, as shown in FIG. 8B, the operator may advance fluid delivery member (162) distally past open distal tip (136) such that a distal portion of fluid delivery member (162) is advanced toward targeted tissue (T). Next, as shown in FIG. 8C, the operator may apply a suitable amount of fluid (F) on targeted tissue (T) to be ablated. The operator may suitably transfer fluid from fluid source (104) onto tissue (T) via fluid delivery member (162) in accordance with the description herein. Fluid (F) is an electrically conductive fluid, such as saline, which may further promote ablation of tissue (T) located between electrode (166, 168). It should be understood that any suitable amount of fluid (F) may be utilized as would be apparent to one skilled in the art in view of the teachings herein.

Next, as shown in FIG. 8D, the operator may further position electrode (166) into suitable placement relative to tissue (T) and fluid (F) for purposes of tissue ablation. In some instances, electrode (166) may be directly touching tissue (T); while in other instances, electrode (166) may be suitably positioned adjacent to tissue (T) (not necessarily contacting tissue (T) directly) and in contact with fluid (F) while the fluid (F) is contacting tissue (T).

Next, as shown in FIG. 8E, the operator may advance probe (164) distally past open distal tip (146) toward targeted tissue (T) for ablation. Probe (164) is positioned such that electrode (168) is suitably positioned relative to tissue (T), fluid (F), and first electrode (166) for purposes of tissue ablation between electrodes (166, 168). It should be understood that at the moment shown in FIG. 8E, the operator may further adjust the spacing between electrodes (166, 168) in accordance with the description herein. Therefore, by adjusting the spacing, the operator may better control the amount of targeted tissue (T) that is to be ablated. As shown in FIG. 8F, with targeted tissue (T) suitably positioned relative to electrodes (166, 168), the operator may activate electrodes (166, 168) to deliver bipolar RF energy to tissue (T) located between electrodes (166, 168), thereby creating a controlled ablation region (A) of targeted tissue (T).

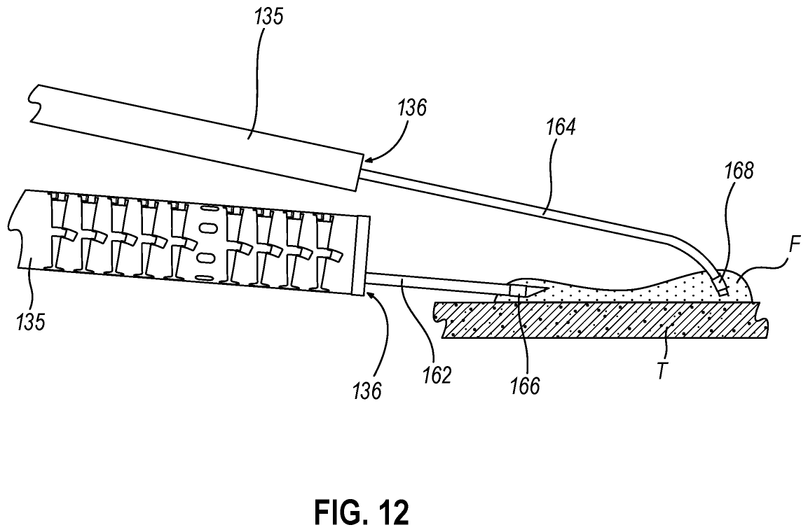

While in the present example, fluid delivery member (162) and probe (164) are contained within a common shaft assembly (130) of a common instrument (300); this is merely optional. FIG. 12 shows an alternative aspect of the disclosure where two shaft assemblies (130) are utilized, each housing either fluid delivery member (162) or probe (164), respectively. Therefore, fluid deliver member (162) and probe (164) may be introduced at different access locations, which may provide for alternative spacing arrangements between electrodes (166, 168).

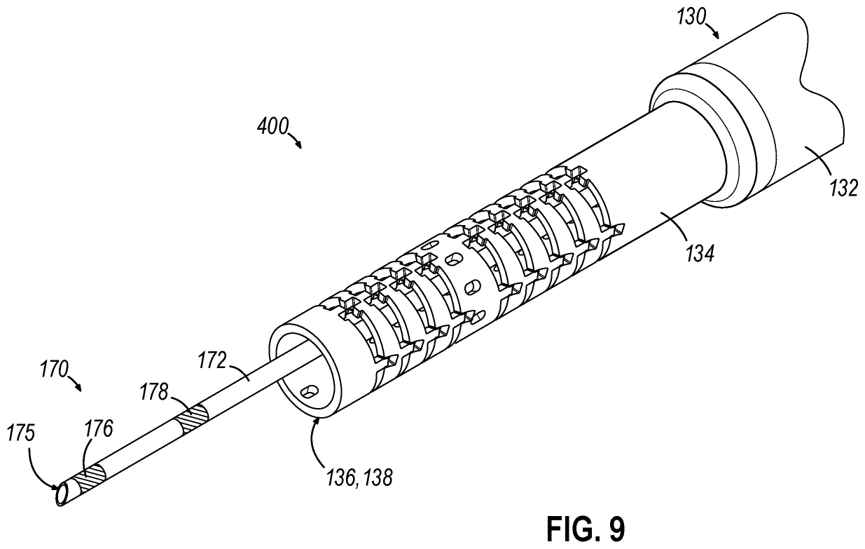

FIG. 9 shows another instrument (400) that may be utilized in replacement to instrument (100, 200, 300) described above. having an alternative electrode assembly (170). Therefore, instrument (400) is substantially similar to instrument (100, 200, 300) described above, except with an alternative electrode assembly (170). In the present example, electrode assembly (170) includes a fluid delivery member (172) defining a fluid lumen (175), that is substantially similar to fluid delivery member (162) described above. However, instead of having a single electrode (166) attached to fluid delivery member (162), fluid delivery member (172) of the present example includes a first electrode (176) and a second electrode (178); which may be substantially similar to electrodes (166, 168) described above, respectively. Therefore, fluid delivery member (172) may be utilized to provide a suitable amount of fluid (F) to a targeted anatomical structure, and also activate electrodes (176, 178) in order to suitably ablate tissue at the targeted anatomical structure.

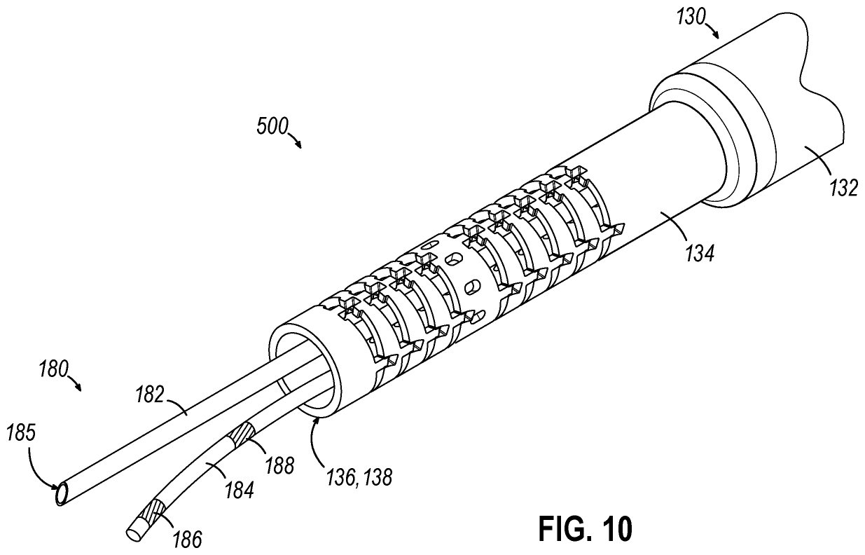

FIG. 10 shows another instrument (500) that may be utilized in replacement to instrument (100, 200, 300, 400) described above. having an alternative electrode assembly (180). Therefore, instrument (500) is substantially similar to instrument (100, 200, 300, 400) described above, except with an alternative electrode assembly (180). In the present example, electrode assembly (180) includes a fluid delivery member (182) defining a fluid lumen (185), and a probe (184); which are substantially similar to fluid delivery member (162), fluid lumen (165), and probe (164) described above, respectively, with differences elaborated below. However, instead of having a single probe (164) with an electrode (168) and another electrode (166) attached to fluid delivery member (162), fluid delivery member (182) of the present example includes no electrodes, while probe (184) includes a first electrode (186) and a second electrode (188); which may be substantially similar to electrodes (166, 168) described above, respectively, except both being attached to probe (184). Therefore, fluid delivery member (182) may be utilized to provide a suitable amount of fluid (F) to a targeted anatomical structure, while probe (184) may be utilized to activate electrodes (186, 188) in order to suitably ablate tissue at the targeted anatomical structure.

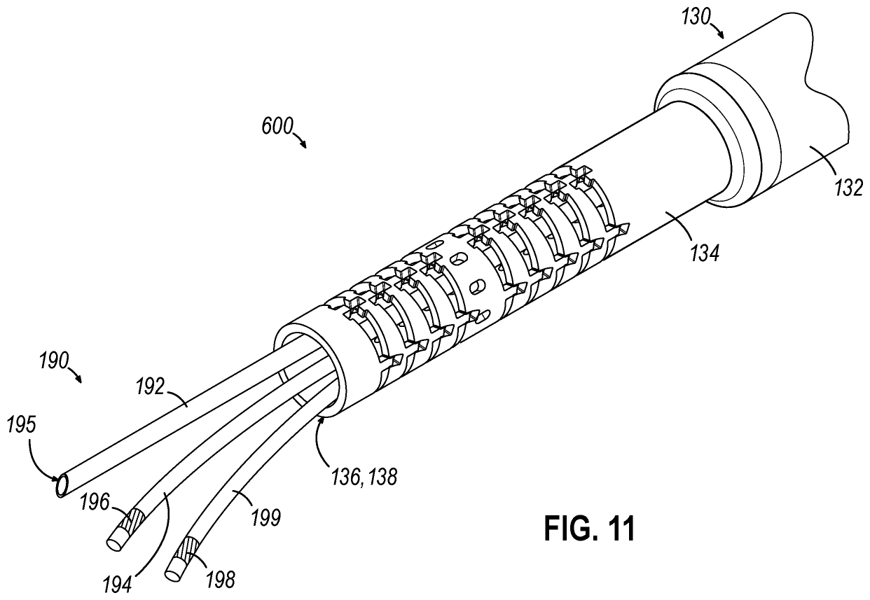

FIG. 11 shows another instrument (600) that may be utilized in replacement to instrument (100, 200, 300, 400, 500) described above. having an alternative electrode assembly (190). Therefore, instrument (600) is substantially similar to instrument (100, 200, 300, 400, 500) described above, except with an alternative electrode assembly (190). In the present example, electrode assembly (190) includes a fluid delivery member (192) defining a fluid lumen (195), and a probe (194); which are substantially similar to fluid delivery member (162), fluid lumen (165), and probe (164) described above, respectively, with differences elaborated below. However, instead of having a single probe (164) with an electrode (168) and another electrode (166) attached to fluid delivery member (162), fluid delivery member (192) of the present example includes no electrodes, while probe (194) includes a first electrode (196), and a second, independently actuating probe (199) includes a second electrode (198); which may be substantially similar to electrodes (166, 168) described above, respectively, except each electrode (196, 198) being attached to an independent probe (194, 199). Therefore, fluid delivery member (182) may be utilized to provide a suitable amount of fluid (F) to a targeted anatomical structure, while probes (194, 199) may be utilized to activate electrodes (196, 198) in order to suitably ablate tissue at the targeted anatomical structure.

While instrument (100, 200, 300, 400, 500, 600) is shown being used to deliver RF energy to tissue for purposes of tissue ablation; it should be understood instrument (100, 200, 300, 400, 500, 600) may be utilized to deliver other forms of energy to tissue and for suitable purposes other than tissue ablation as would be apparent to one skilled in the art in view of the teachings herein. For example, rather than applying RF energy, instrument (100, 200, 300, 400, 500, 600) may be operative to apply pulsed field DC energy, such as in an irreversible electroporation (IRE) procedure.

### II. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A method comprising: (a) inserting a variable distance electrode assembly within a patient; (b) placing a first electrode of the variable distance electrode assembly adjacent to a targeted tissue area; (c) placing a second electrode of the variable distance electrode assembly adjacent to the targeted tissue area such that the first electrode and the second electrode are spaced from each other to define a first distance; (d) activating the first electrode and the second electrode to apply electrical energy to the targeted tissue area; and (e) translating the first electrode relative to the second electrode to define a second distance between each other while the first electrode and the second electrode continue to apply electrical energy to the targeted tissue area.

### Example 2

The method of Example 1, further comprising inserting a shaft assembly containing the variable distance electrode assembly within the patient while inserting the variable distance electrode assembly.

### Example 3

The method of Example 2, the first electrode being fixed to the shaft assembly.

### Example 4

The method of Example 3, the first electrode being annular shaped and fixed to an open distal end of the shaft assembly.

### Example 5

The method of any of Examples 3 through 4, the second electrode being connected to an actuatable body that is slidably coupled with the shaft assembly, the method further comprising translating the actuating body relative to the shaft assembly while placing the second electrode adjacent to the targeted tissue area.

### Example 6

The method of Example 5, further comprising translating the second electrode distally relative to the first electrode while placing the second electrode adjacent to the targeted tissue area.

### Example 7

The method of Example 6, further comprising dragging at least one of the first electrode or the second electrode against the targeted tissue area while translating the first electrode relative to the second electrode to define the second distance.

### Example 8

The method of any of Examples 1 through 7, further comprising communicating a fluid to the targeted tissue area.

### Example 9

The method of Example 8, further comprising actuating a fluid delivery member toward the targeted tissue area.

### Example 10

The method of Example 9, the fluid delivery member being slidably disposed within a shaft assembly, the method further comprising actuating the fluid delivery member distally past the shaft assembly while actuating the fluid delivery member within the anatomical passageway.

### Example 11

The method of Example 10, the fluid delivery member further comprising a needle, the method further comprising penetrating tissue with the needle.

### Example 12

The method of any of Examples 10 through 11, the first electrode being attached to the fluid delivery member.

### Example 13

The method of Example 12, the second electrode being attached to a probe, the

probe being actuatable relative to the fluid delivery member.

### Example 14

The method of Example 10, the first electrode being attached to a first probe.

### Example 15

The method of Example 14, the second electrode being attached to a second probe that is actuatable relative to the first probe.

### Example 16

The method of any of Examples 1 through 15, further comprising ablating the targeted tissue area while activating the first electrode and the second electrode.

### Example 17

The method of any of Examples 1 through 16, further comprising applying RF energy to ablate the targeted tissue area while activating the first electrode and the second electrode.

### Example 18

An apparatus, comprising: (a) a shaft assembly defining a working lumen and terminating into a distal open end; and (b) a variable displacement electrode assembly configured to ablate tissue, comprising: (i) a first electrode, and (ii) a second electrode configured to translate relative to the shaft assembly and the first electrode, the first electrode and the second electrode configured to cooperatively ablate tissue while the second electrode and the first electrode translate relative to each other while directly engaging tissue.

### Example 19

The apparatus of Example 18, the second electrode being attached to an actuatable body slidably contained within the shaft assembly.

### Example 20

The apparatus of any of Examples 18 through 19, the first electrode being fixed to the open distal end of the shaft assembly.

### Example 21

The apparatus of any of Examples 18 through 20, further comprising a handle assembly, the handle assembly comprising: (a) a body, (b) a first slider, slidably attached to the body and operatively engaged with the actuatable body.

### Example 22

An apparatus, comprising: (a) a shaft assembly defining a working lumen and terminating into a distal open end; and (b) a variable displacement electrode assembly configured to ablate tissue, comprising: (i) a first electrode, and (ii) a second electrode configured to translate relative to the shaft assembly and the first electrode, the first electrode or the second electrode configured to drag across tissue while cooperatively ablating tissue to define an elongated ablation section.

### III. Miscellaneous

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A method comprising:
(a) inserting a variable distance electrode assembly within a patient;
(b) placing a first electrode of the variable distance electrode assembly adjacent to a targeted tissue area;
(c) placing a second electrode of the variable distance electrode assembly adjacent to the targeted tissue area such that the first electrode and the second electrode are spaced from each other to define a first distance;
(d) activating the first electrode and the second electrode to apply electrical energy to the targeted tissue area; and
(e) translating the first electrode relative to the second electrode to define a second distance between each other while the first electrode and the second electrode continue to apply electrical energy to the targeted tissue area.

2. The method of claim 1, further comprising inserting a shaft assembly containing the variable distance electrode assembly within the patient while inserting the variable distance electrode assembly.

3. The method of claim 2, the first electrode being fixed to the shaft assembly.

4. The method of claim 3, the first electrode being annular shaped and fixed to an open distal end of the shaft assembly.

5. The method of claim 3, the second electrode being connected to an actuatable body that is slidably coupled with the shaft assembly, the method further comprising translating the actuating body relative to the shaft assembly while placing the second electrode adjacent to the targeted tissue area.

6. The method of claim 5, further comprising translating the second electrode distally relative to the first electrode while placing the second electrode adjacent to the targeted tissue area.

7. The method of claim 6, further comprising dragging at least one of the first electrode or the second electrode against the targeted tissue area while translating the first electrode relative to the second electrode to define the second distance.

8. The method of claim 1, further comprising communicating a fluid to the targeted tissue area.

9. The method of claim 8, further comprising actuating a fluid delivery member toward the targeted tissue area.

10. The method of claim 9, the fluid delivery member being slidably disposed within a shaft assembly, the method further comprising actuating the fluid delivery member distally past the shaft assembly while actuating the fluid delivery member within the anatomical passageway.

11. The method of claim 10, the fluid delivery member further comprising a needle, the method further comprising penetrating tissue with the needle.

12. The method of claim 10, the first electrode being attached to the fluid delivery member.

13. The method of claim 12, the second electrode being attached to a probe, the probe being actuatable relative to the fluid delivery member.

14. The method of claim 10, the first electrode being attached to a first probe.

15. The method of claim 14, the second electrode being attached to a second probe that is actuatable relative to the first probe.

16. The method of claim 1, further comprising applying RF energy to ablate the targeted tissue area while activating the first electrode and the second electrode.

17. An apparatus, comprising:
(a) a shaft assembly defining a working lumen and terminating into a distal open end; and
(b) a variable displacement electrode assembly configured to ablate tissue, comprising:
(i) a first electrode, and
(ii) a second electrode configured to translate relative to the shaft assembly and the first electrode, the first electrode and the second electrode configured to cooperatively ablate tissue while the second electrode and the first electrode translate relative to each other while directly engaging tissue.

18. The apparatus of claim 17, the second electrode being attached to an actuatable body slidably contained within the shaft assembly.

19. The apparatus of claim 17, further comprising a handle assembly, the handle assembly comprising:
(a) a body,
(b) a first slider, slidably attached to the body and operatively engaged with the actuatable body.

20. An apparatus, comprising:
(a) a shaft assembly defining a working lumen and terminating into a distal open end; and
(b) a variable displacement electrode assembly configured to ablate tissue, comprising:
(i) a first electrode, and
(ii) a second electrode configured to translate relative to the shaft assembly and the first electrode, the first electrode or the second electrode configured to drag across tissue while cooperatively ablating tissue to define an elongated ablation section.
